(19) United States
(12) Patent Application Publication
(10) Pub. No.: US 2022/0153744 A1
(43) Pub. Date: May 19, 2022

(54) SOLID STATE FORMS OF ACALABRUTINIB

(71) Applicant: ASSIA CHEMICAL INDUSTRIES LTD., Petach Tikva (IL)

(72) Inventors: Sundara Lakshmi Kanniah, Vellore (IN); Anantha Rajmohan Muthusamy, Sivakasi (IN); Bhupendra Prakash Tyagi, Greater Noida (IN); Vrajlal Karamshibhai Gothalia, Greater Noida (IN); Sanjay Jaiswal, Greater Noida (IN); Sadanand Hardeo Maurya, Thane (IN); Parven Kumar Luthra, New Delhi (IN); Amit Gupta, Ghaziabad (IN); Manigandan Gopal, Greater Noida (IN); Shilpi Pandey, Greater Noida (IN)

(21) Appl. No.: 17/442,302

(22) PCT Filed: Mar. 26, 2020
(86) PCT No.: PCT/US20/24877

§ 371 Date: Sep. 23, 2021

(30) Foreign Application Priority Data
Mar. 27, 2019 (IN) 201911011869
May 31, 2019 (IN) 201911021770
Feb. 6, 2020 (IN) 202011005126
Feb. 27, 2020 (IN) 202011008280

(51) Int. Cl. C07D 487/04; A61P 35/02; C07D 487/16

(52) U.S. Cl. CPC C07D 487/04; C07B 2200/13; C07D 487/16; A61P 35/02

(57) ABSTRACT

The present disclosure relates to solid state forms of Acalabrutinib, processes for the preparation thereof and pharmaceutical compositions comprising said solid state forms of Acalabrutinib.

### FIELD OF THE INVENTION

The present disclosure relates to solid state forms of Acalabrutinib, processes for the preparation thereof and pharmaceutical compositions comprising said solid state forms of Acalabrutinib.

The present disclosure also relates to (S)-4-(9-(1-(but-2-ynoyl)pyrrolidin-2-yl)-4-methyl-2-oxo-2H-imidazo[5′,1′:3,4]pyrazino[1,2-a]pyrimidin-11-yl)-N-(pyridin-2-yl)benzamide [“Compound 1” ], which is an impurity of Acalabrutinib, and to processes for its preparation. The present disclosure further relates to compositions comprising amorphous Acalabrutinib and having the respective impurity (Compound 1) at a level of less than about 0.2%, or from about 0.02% to about 0.2%.

### BACKGROUND OF THE INVENTION

Acalabrutinib has the chemical name 4-{8-Amino-3-[(2S)-1-(2-butynoyl)-2-pyrrolidinyl]imidazo[1,5-a]pyrazin-1-yl}-N-(2-pyridinyl)benzamide. Acalabrutinib has the following chemical structure:

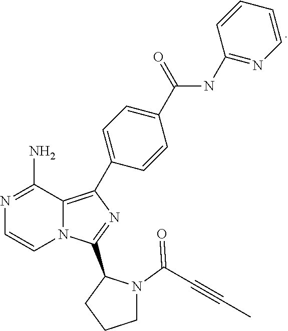 [SMILES: CC#CC(=O)N1CCC[C@H]1c1nc(-c2ccc(C(=O)Nc3ccccn3)cc2)c2c(N)nccn12]

Acalabrutinib is being developed for the treatment of hematologic diseases like chronic lymphocytic leukaemia (CLL), mantle cell lymphoma (MCL) and lymphoplasmacytic lymphoma (Waldenström's macroglobulinaemia, WM).

Acalabrutinib is disclosed in WO 2013/010868 (referred to as '868). According to the '868 applicant, as well as statements in later publications, the procedure disclosed in this publication has been found to produce Acalabrutinib in an amorphous form. WO 2017/002098 discloses amorphous and crystalline forms of Acalabrutinib, as well as Acalabrutinib salts. According to this publication, the product obtained in WO 2013/010868 is an amorphous form. Moreover, the Acalabrutinib product was found to have a tendency to form an oil. WO 2019/041026 and WO 2018/064797 also disclose solid state forms and co-crystals of Acalabrutinib.

Polymorphism, the occurrence of different crystal forms, is a property of some molecules and molecular complexes. A single compound, like Acalabrutinib, may give rise to a variety of polymorphs having distinct crystal structures and physical properties like melting point, thermal behavior (e.g. measured by thermogravimetric analysis—“TGA”, or differential scanning calorimetry—“DSC”), X-ray powder diffraction (XRPD, or sometimes also referred to as PXRD) pattern, infrared absorption fingerprint, Raman absorption fingerprint, and solid state (¹³C-) NMR spectrum. One or more of these techniques may be used to distinguish different polymorphic forms of a compound.

Different solid state forms (including solvated forms) of an active pharmaceutical ingredient may possess different properties. Such variations in the properties of different solid state forms and solvates may provide a basis for improving processing or its formulation into a pharmaceutical product, for example, by facilitating better processing or handling characteristics, improving the dissolution profile, or improving stability (polymorph as well as chemical stability) and shelf-life. These variations in the properties of different solid state forms may also provide improvements to the final dosage form, for instance, if they serve to improve bioavailability. Different solid state forms and solvates of an active pharmaceutical ingredient may also give rise to a variety of polymorphs or crystalline forms, which may in turn provide additional opportunities to use variations in the properties and characteristics of a solid active pharmaceutical ingredient for providing an improved product.

Discovering new solid state forms and solvates of a pharmaceutical product can provide materials having desirable processing properties, such as ease of handling, ease of processing, storage stability, and ease of purification, or as desirable intermediate crystal forms that facilitate conversion to other polymorphic forms. New polymorphic forms and solvates of a pharmaceutically useful compound can also provide an opportunity to improve the performance characteristics of a pharmaceutical product (dissolution profile, bioavailability, etc.). It enlarges the repertoire of materials that a formulation scientist has available for formulation optimization, for example by providing a product with different properties, e.g., a different crystal habit, higher crystallinity or polymorphic stability which may offer better processing or handling characteristics, improved dissolution profile, or improved shelf-life.

For at least these reasons, there is a need for additional solid state forms (including solvated forms) of Acalabrutinib.

In addition, identifying impurities and controlling formation of such impurities is of importance for development and manufacture of pharmaceutical compounds. The present disclosure identifies an impurity of Acalabrutinib, and an amorphous form of Acalabrutinib comprising said impurity.

### SUMMARY OF THE INVENTION

The present disclosure generally relates to solid state forms of Acalabrutinib, processes for their preparation, and pharmaceutical compositions comprising these solid state forms.

In one aspect, the present disclosure relates to a crystalline Form ACB3 of Acalabrutinib, characterized by data selected from one or more of the following:
a. an XRPD pattern having peaks at 6.3, 16.3, 17.5, 18.5, 19.6 and 24.0 degrees 2-theta±0.2 degrees 2-theta;
b. an XRPD pattern as depicted in FIG. 4;
c. a ¹³C solid state NMR having peaks in the range of 100-200 ppm at 107.0, 113.8, 137.8, 141.9, 146.5 and 165.4 ppm+0.2 ppm;
d. a solid state ¹³C NMR spectrum having absolute chemical shift differences from a reference peak at 127.3±2 ppm of 20.3, 13.5, 10.5, 14.6, 19.2 and 38.1 ppm+0.1 ppm respectively;
e. a ¹³C solid state NMR spectrum substantially as depicted in FIG. 6a, 6b or 6c; and/or
f. combinations of these data.

The crystalline Form ACB3 of Acalabrutinib of the present disclosure may in some embodiments be an anhydrous form.

In another aspect, the present disclosure encompasses the use of the described solid state forms of Acalabrutinib, in particular of form ACB3, for the preparation of pharmaceutical compositions and/or pharmaceutical formulations. Such compositions and formulations are in some embodiments suitable for the treatment of hematologic diseases, such as forms of blood cancers.

Accordingly, the present disclosure further provides pharmaceutical compositions comprising any one or a combination of the solid state forms of Acalabrutinib according to the present disclosure.

In yet another aspect, the present disclosure also encompasses pharmaceutical formulations comprising any one or a combination of the described solid state forms of Acalabrutinib, or a pharmaceutical composition comprising any one or a combination of the solid state forms of Acalabrutinib according to the present disclosure, and at least one pharmaceutically acceptable excipient.

The present disclosure further encompasses processes to prepare said pharmaceutical formulations of Acalabrutinib comprising combining any one or a combination of the described solid state forms with at least one pharmaceutically acceptable excipient.

The solid state forms defined herein as well as the pharmaceutical compositions or formulations of the solid state form of Acalabrutinib can be used as medicaments. In some embodiments, the solid state forms described herein as well as the pharmaceutical compositions or formulations of the solid state forms of Acalabrutinib can be used for the treatment of hematologic diseases, such as forms of blood cancers. Examples of such blood cancers include chronic lymphocytic leukaemia (CLL), mantle cell lymphoma (MCL) and lymphoplasmacytic lymphoma (Waldenstrom's macroglobulinaemia, WM).

In a related aspect, the present disclosure also provides methods of treating hematologic diseases, such as forms of blood cancers; comprising administering a therapeutically effective amount of any one or a combination of the described solid state forms, or at least one of the herein described pharmaceutical compositions or formulations, to a subject suffering from said hematologic diseases (including said forms of blood cancers), or otherwise in need of the treatment.

The present disclosure also provides uses of the solid state forms of Acalabrutinib described herein for preparing other solid state forms of Acalabrutinib and/or Acalabrutinib co-crystals and/or salts, and their solid state forms.

The present disclosure further provides processes for preparing other solid state forms of Acalabrutinib and/or Acalabrutinib co-crystals and/or salts, and their solid state forms thereof.

The processes for preparing an Acalabrutinib salt or a solid state form thereof comprise preparing the solid state forms of Acalabrutinib as described herein, such as crystalline Form ACB3 of Acalabrutinib, and converting it to an Acalabrutinib salt, co-crystal or a solid state form thereof. The process may optionally further comprise combining the resulting Acalabrutinib salt, co-crystal or a solid state form thereof, with at least one pharmaceutically acceptable excipient to prepare a pharmaceutical composition or formulation.

The present disclosure further relates to a compound named (S)-4-(9-(1-(but-2-ynoyl)pyrrolidin-2-yl)-4-methyl-2-oxo-2H-imidazo[5′,1′:3,4]pyrazino[1,2-a]pyrimidin-11-yl)-N-(pyridin-2-yl)benzamide (hereinafter also referred to as “Compound 1”), which is an impurity of Acalabrutinib, and to processes for the preparation of Compound 1.

The present disclosure also relates to compositions comprising amorphous Acalabrutinib and having Compound 1 at a level of less than about 0.2%; or less than about 0.15%; or less than about 0.1% by weight.

In some embodiments Compound 1 is present at a level of from about 0.02% to about 0.2%; or from about 0.02% to about 0.15%; or from about 0.02% to about 0.1% by weight.

In other embodiments, Compound 1 is present at a level of from about 0.05% to about 0.2%; or from about 0.05% to about 0.15%; or from about 0.05% to about 0.1% by weight.

In another aspect, the present disclosure relates to amorphous Acalabrutinib forming Compound 1 at a level of less than about 0.2%; or less than about 0.15%; or less than about 0.1% by weight, when stored at a temperature of about 25° C. and relative humidity (“RH”) of about 60% for a period of 1 month, or for a period of 3 months, or for a period of 6 months.

In some embodiments, amorphous Acalabrutinib forms Compound 1 at a level of from about 0.02% to about 0.2%; or from about 0.02% to about 0.15%; or from about 0.02% to about 0.1% by weight, when stored at a temperature of about 25° C. and RH of about 60% for a period of 1 month, or for a period of 3 months, or for a period of 6 months.

In other embodiments, amorphous Acalabrutinib forms Compound 1 at a level of from about 0.05% to about 0.2%; or from about 0.05% to about 0.15%; or from about 0.05% to about 0.1% by weight, when stored at a temperature of about 25° C. and RH of about 60% for a period of 1 month, or for a period of 3 months, or for a period of 6 months.

### BRIEF DESCRIPTION OF THE FIGURES

FIG. 1 shows an X-ray powder diffractogram (XRPD) of form ACB1 of Acalabrutinib.

FIG. 2 shows an XRPD of form ACB2 of Acalabrutinib.

FIG. 3 shows an XRPD of form III of Acalabrutinib, as described in WO 2017/002095.

FIG. 4 shows an XRPD of form ACB3 of Acalabrutinib.

FIG. 5 shows an XRPD of form ACB4 of Acalabrutinib.

FIG. 6a shows a ¹³C solid state NMR spectrum of form ACB3 of Acalabrutinib (Full scan).

FIG. 6b shows a ¹³C solid state NMR spectrum of form ACB3 of Acalabrutinib (at the range of 0-100 ppm).

FIG. 6c shows a ¹³C solid state NMR spectrum of form ACB3 of Acalabrutinib (at the range of 100-200 ppm).

FIG. 7 shows a ¹H NMR of Compound 1.

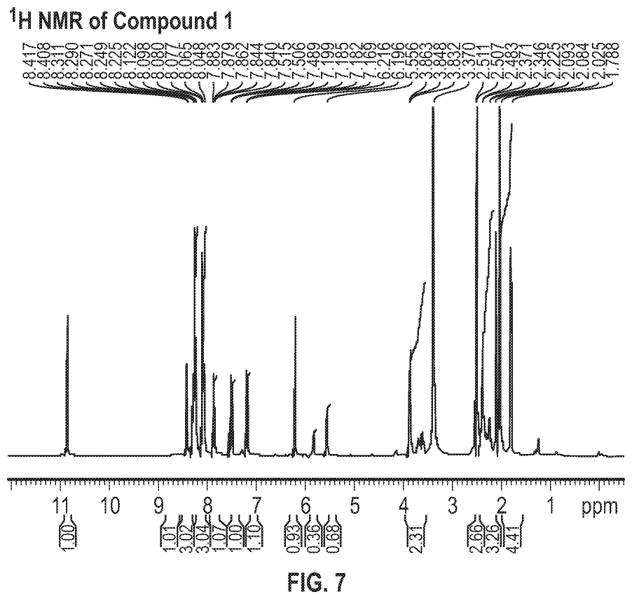

FIG. 8 shows a ¹³C NMR of Compound 1.

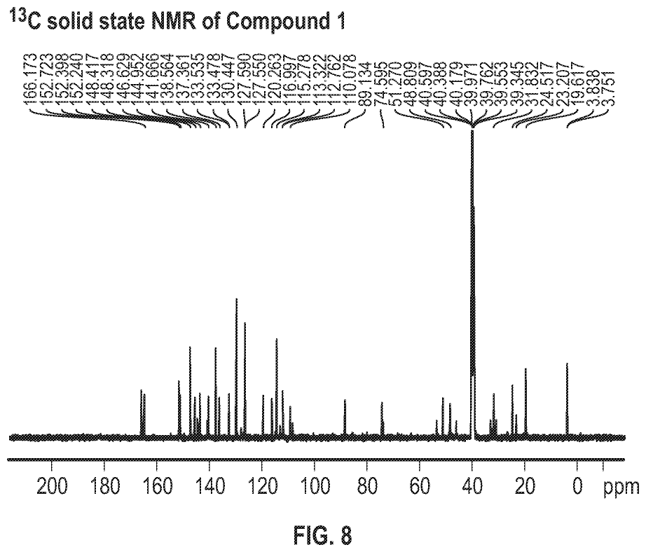

FIG. 9 shows a mass spectrum of Compound 1.

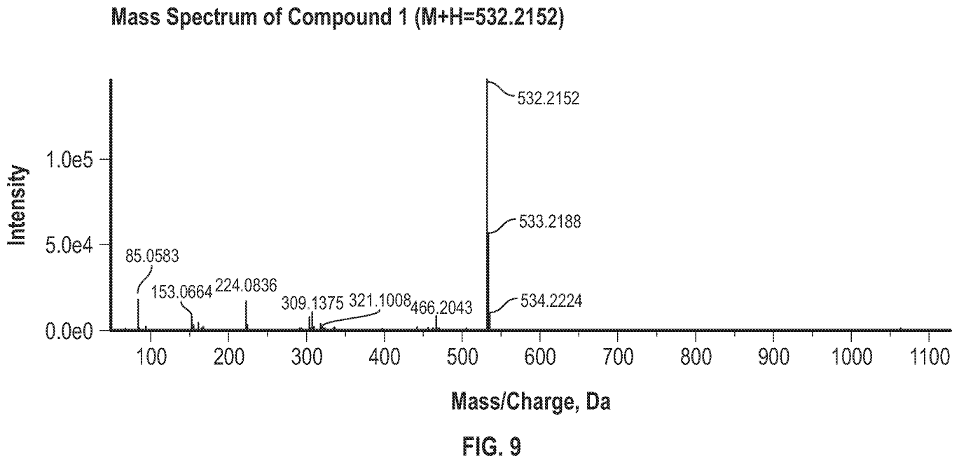

### DETAILED DESCRIPTION OF THE INVENTION

The present disclosure relates to solid state forms of Acalabrutinib, processes for their preparation, and pharmaceutical compositions comprising these solid state forms.

In addition, the present disclosure also relates to (S)-4-(9-(1-(but-2-ynoyl)pyrrolidin-2-yl)-4-methyl-2-oxo-2H-imidazo[5′,1′:3,4]pyrazino[1,2-a]pyrimidin-11-yl)-N-(pyridin-2-yl)benzamide, which is an impurity of Acalabrutinib and to processes for its preparation. The present disclosure further relates to compositions comprising amorphous Acalabrutinib and containing the respective impurity at a level of less than about 0.2%, or from about 0.02% to about 0.2%.

The process described in WO 2013/010868 (referred to as '868) may afford Acalabrutinib in amorphous form. WO 2017/002095 describes several polymorphs of Acalabrutinib, and also confirms the difficulty in obtaining a crystalline product. The '868 applicant's attempts to prepare a crystalline form of Acalabrutinib by common concentration of API solutions in organic solvents failed, and resulted in a viscous oil product, which finally, upon extended evaporation, converted to an amorphous solidified foam.

The present inventors succeeded in developing an adequate crystallization technique for Acalabrutinib, providing pharmaceutically suitable solid-state forms of Acalabrutinib.

Depending on which solid state form of Acalabrutinib it is compared to, the solid state forms of Acalabrutinib according to the present disclosure may have advantageous properties selected from at least one of: chemical or polymorphic purity, flowability, solubility, wettability, low hygroscopicity, low solvent (e.g. water) content, dissolution rate, bioavailability, morphology or crystal habit, stability—such as chemical stability as well as thermal and mechanical stability with respect to polymorphic conversion, stability towards dehydration and/or storage stability, a lower degree of hygroscopicity, low content of residual solvents and advantageous processing and handling characteristics such as compressibility, or bulk density.

A crystal form may be referred to herein as being characterized by graphical data “as depicted in” a Figure. Such data include, for example, powder X-ray diffractograms and solid state NMR spectra. As is well-known in the art, the graphical data potentially provides additional technical information to further define the respective solid state form (a so-called “fingerprint”) which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Acalabrutinib referred to herein as being characterized by graphical data “as depicted in” a Figure will thus be understood to include any crystal forms of the Acalabrutinib, characterized with the graphical data having such small variations, as are well known to the skilled person, in comparison with the Figure.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or no detectable amount of any other forms of the subject compound as measured, for example, by XRPD. Thus, the solid state form of Acalabrutinib described herein as substantially free of any other solid state forms would be understood to contain greater than about 80% (w/w), greater than about 90% (w/w), greater than about 95% (w/w), greater than about 98% (w/w), greater than about 99% (w/w), or 100% of the subject solid state form of Acalabrutinib.

As used herein, unless stated otherwise, XRPD peaks reported herein have been measured using CuK_{α} radiation, λ=1.5418 Å.

As used herein, the term “isolated” in reference to solid state forms of Acalabrutinib of the present disclosure corresponds to solid state form of Acalabrutinib that is physically separated from the reaction mixture in which it is formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., about 22° C. to about 27° C., or about 25° C.

As used herein, unless indicated otherwise, the term “elevated temperature” refers to any temperature above room temperature, preferably above about 20° C., and more preferably above about 25° C.

A process or step may be referred to herein as being carried out “overnight.” This refers to a time interval, e.g., for the process or step, that spans the time during the night, when that process or step may not be actively observed. This time interval is from about 8 to about 20 hours, about 10 to about 18 hours, or about 16 hours.

As used herein, and unless stated otherwise, the term “anhydrous” in relation to crystalline Acalabrutinib relates to a crystalline Acalabrutinib which does not include any crystalline water (or other solvents) in a defined, stoichiometric amount within the crystal. Moreover, an “anhydrous” form does not contain more than 1% (w/w) of either water or organic solvents as measured for example by TGA.

The term “solvate”, as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding methyl tert-butyl ether (MTBE) (1.5 v/v) to a 100 ml reaction mixture would indicate that 150 mL of MTBE was added.

As used herein, the term “reduced pressure” refers to a pressure of from about 10 pbar to 50 mbar.

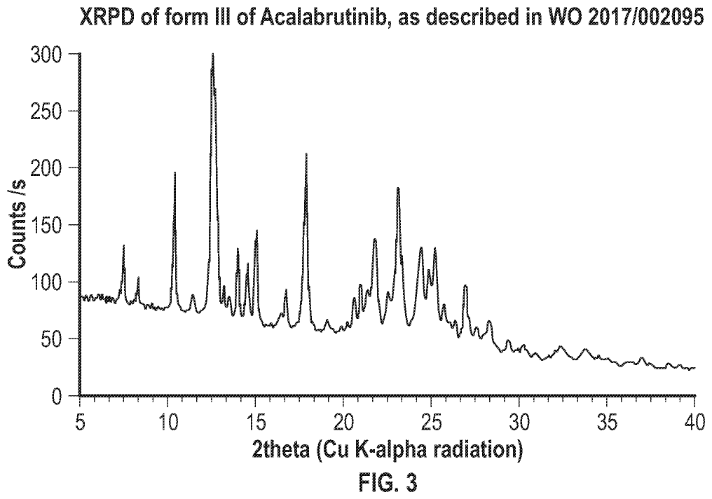

As used herein, and unless stated otherwise, the term Acalabrutinib Form III relates to Form III as described in WO 2017/002095. Form III can for example be described by the XRPD pattern as presented in FIG. 3.

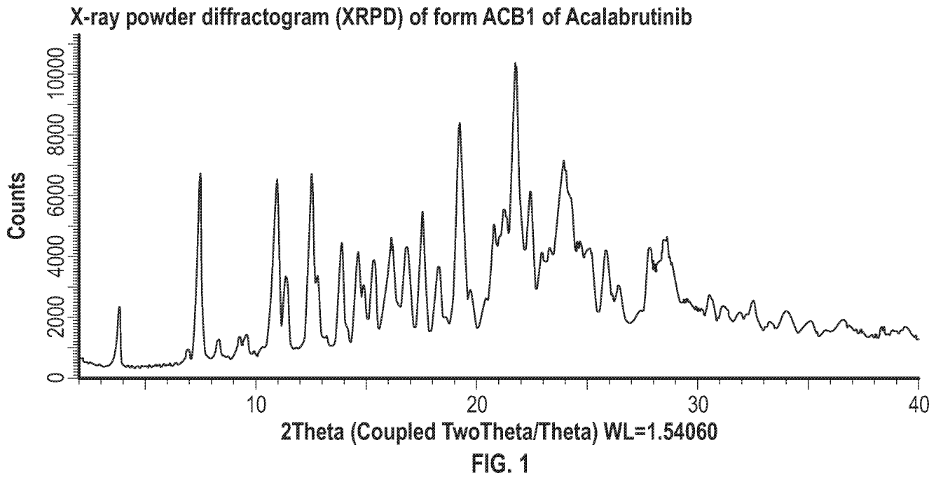

The present disclosure includes a crystalline form of Acalabrutinib designated as Form ACB1. The crystalline Form ACB1 of Acalabrutinib can be characterized by data selected from one or more of the following: an XRPD pattern having peaks at 3.7, 7.4, 13.9, 16.1, 18.2 and 19.2 degrees 2-theta±0.2 degrees 2-theta; an XRPD pattern as depicted in FIG. 1; and combinations of these data.

Crystalline Form ACB1 of Acalabrutinib may in some embodiments be further characterized by the XRPD pattern having peaks at 3.7, 7.4, 13.9, 16.1, 18.2 and 19.2 degrees 2-theta±0.2 degrees 2-theta, and also having one, two, three, four, five or six additional peaks selected from 10.9, 12.5, 14.7, 15.3, 21.7 and 24.0 degrees two theta±0.2 degrees two theta.

Crystalline Form ACB1 of Acalabrutinib may be characterized by each of the above characteristics alone/or by all possible combinations, e.g. by XRPD pattern having peaks at 3.7, 7.4, 13.9, 16.1, 18.2 and 19.2 degrees 2-theta±0.2 degrees 2-theta and/or an XRPD pattern as depicted in FIG. 1.

Crystalline Form ACB1 of Acalabrutinib may be prepared by a process comprising crystallization of Acalabrutinib from a mixture comprising ethanol as a solvent and n-heptane as an anti-solvent. The crystallization comprises providing a solution of Acalabrutinib in ethanol and combining the solution with n-heptane to obtain a suspension.

Typically, the solution is provided at a temperature of from about 20° C. to about 50° C., preferably from about 20° C. to about 30° C. or from about 30° C. to about 50° C.

Combining the solution with the anti-solvent can be done either by direct addition, i.e. the anti-solvent is added to the solution; or by reverse addition, i.e., the solution is added to the anti-solvent.

The process for preparing crystalline Form ACB1 of Acalabrutinib may further comprise recovering said crystalline form. The recovery may be done, for example, by filtering the suspension, for example by vacuum filtration; optionally washing; and drying. Preferably, drying is done by air, typically at room temperature.

Crystalline Form ACB1 of Acalabrutinib may also be prepared by a process comprising precipitating Form ACB1 from a slurry of ethanol and methyl tert-butyl ether (“MTBE”).

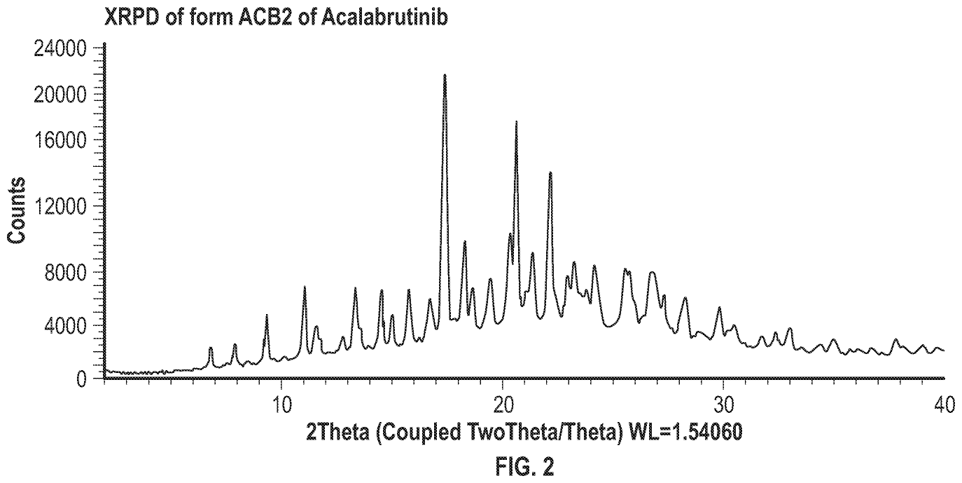

The present disclosure further includes a crystalline form of Acalabrutinib designated as Form ACB2. The crystalline Form ACB2 of Acalabrutinib can be characterized by data selected from one or more of the following: an XRPD pattern having peaks at 7.7, 9.2, 10.9, 15.6, 16.5 and 17.2 degrees 2-theta±0.2 degrees 2-theta; an XRPD pattern as depicted in FIG. 2; and combinations of these data.

Crystalline Form ACB2 of Acalabrutinib may in some embodiments be further characterized by the XRPD pattern having peaks at 7.7, 9.2, 10.9, 15.6, 16.5 and 17.2 degrees 2-theta±0.2 degrees 2-theta, and also having one, two, three, four or five additional peaks selected from 13.2, 18.1, 20.5, 21.2 and 22.0 degrees two theta±0.2 degrees two theta.

Crystalline Form ACB2 of Acalabrutinib may be characterized by each of the above characteristics alone/or by all possible combinations, e.g. by XRPD pattern having peaks at 7.7, 9.2, 10.9, 15.6, 16.5 and 17.2 degrees 2-theta±0.2 degrees 2-theta and/or an XRPD pattern as depicted in FIG. 2.

Crystalline Form ACB2 of Acalabrutinib may be a hydrate form, and acetonitrile solvate or hydrate-acetonitrile solvate. The water and solvent content may be from about 1.5% to about 5% (w/w), as measured by typical methods, such as TGA.

Crystalline Form ACB2 of Acalabrutinib may be prepared a process comprising crystallization of Form ACB2 from acetonitrile. Typically, the crystallization is done without presence of water, preferably the moisture content in the acetonitrile solvent used, is in amount of less than 1% (w/w). Alternatively, the crystallization may be done with a mixture of acetonitrile and water, which may result in Crystalline Form ACB2 of Acalabrutinib in either hydrate or hydrate-solvate form.

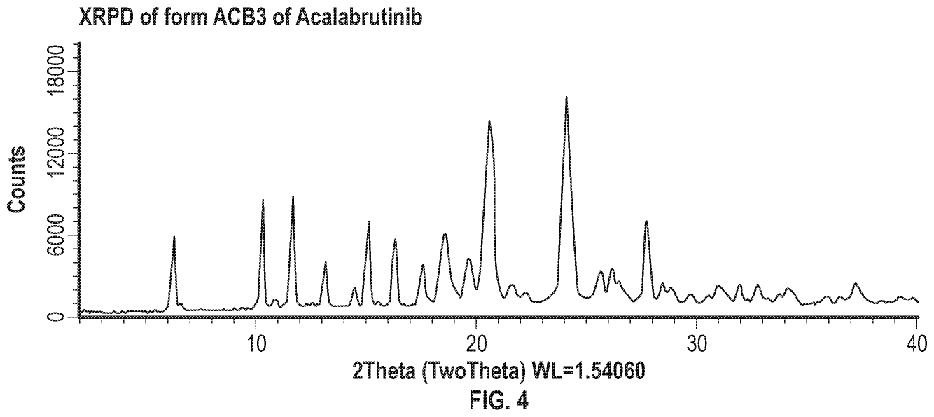

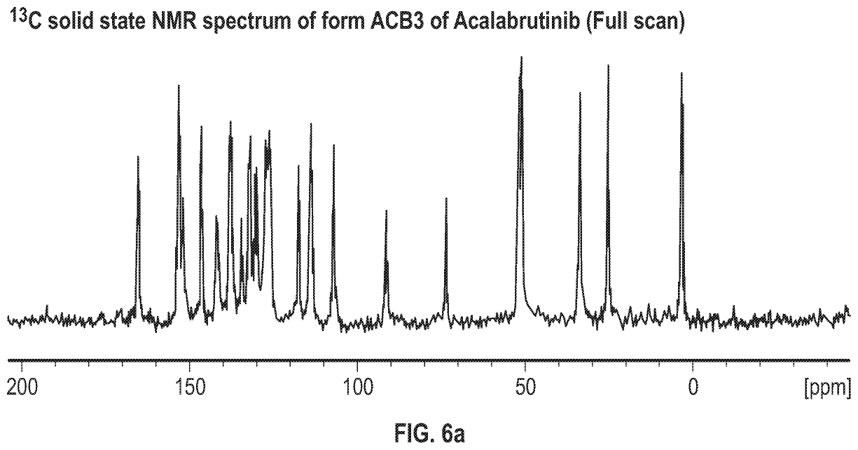

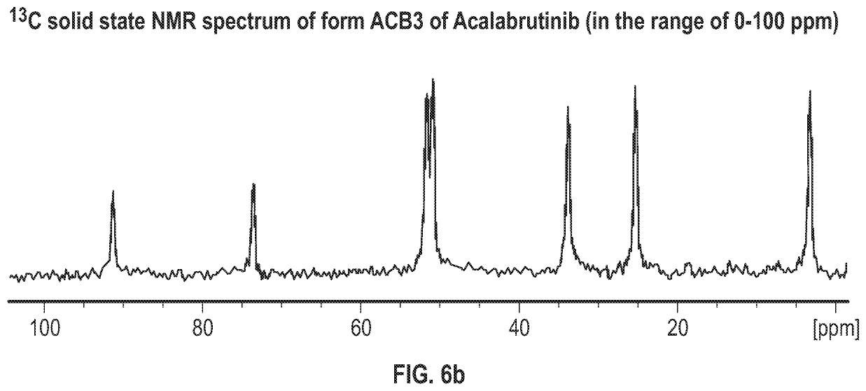

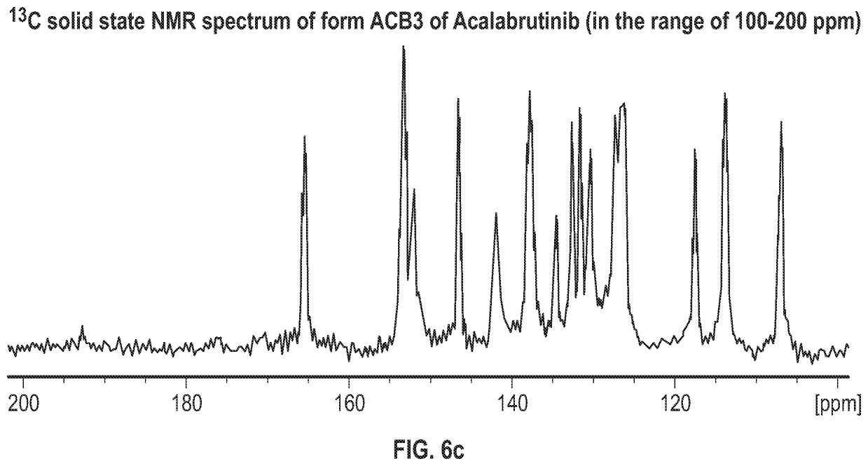

The present disclosure further includes a crystalline form of Acalabrutinib designated as Form ACB3. The crystalline Form ACB3 of Acalabrutinib can be characterized by data selected from one or more of the following: an XRPD pattern having peaks at 6.3, 16.3, 17.5, 18.5, 19.6 and 24.0 degrees 2-theta±0.2 degrees 2-theta; an XRPD pattern as depicted in FIG. 4; a ¹³C solid state NMR having peaks in the range of 100-200 ppm at 107.0, 113.8, 137.8, 141.9, 146.5 and 165.4 ppm±0.2 ppm; a solid state ¹³C NMR spectrum having the following chemical shift absolute differences from a reference peak at 127.3±2 ppm of 20.3, 13.5, 10.5, 14.6, 19.2 and 38.1 ppm±0.1 ppm respectively; a ¹³C solid state NMR spectrum substantially as depicted in FIG. 6a, 6b or 6c; and combinations of these data.

Crystalline Form ACB3 of Acalabrutinib may in some embodiments be further characterized by the XRPD pattern having peaks at 6.3, 16.3, 17.5, 18.5, 19.6 and 24.0 degrees 2-theta±0.2 degrees 2-theta, and also having one, two, three, four or five additional peaks selected from 10.3, 13.1, 15.1, 20.5 and 27.7 degrees two theta±0.2 degrees two theta.

In some embodiments, crystalline Form ACB3 of Acalabrutinib may be an anhydrous form.

Crystalline Form ACB3 of Acalabrutinib may be characterized by each of the above characteristics alone/or by all possible combinations, e.g. by XRPD pattern having peaks at 6.3, 16.3, 17.5, 18.5, 19.6 and 24.0 degrees 2-theta±0.2 degrees 2-theta and/or an XRPD pattern as depicted in FIG. 4.

Crystalline Form ACB3 of Acalabrutinib may have advantageous properties as described herein above. Particularly, Form ACB3 is polymorphically stable under various thermodynamic and/or physical conditions. For example, Form ACB3 is polymorphically stable when stored at room temperature and relative humidity (“RH”) of about 80% for a period of at least 7 days, or at room temperature and RH of about 60% for a period of at least 1 month, or at temperature of 40° C. and RH of about 75% for a period of at least 1 month. In addition, it remains polymorphically stable when heated to a temperature of about 100° C. over a period of about 30 minutes. It is also polymorphically stable towards solvent grinding or strong dry grinding.

Crystalline Form ACB3 of Acalabrutinib may be prepared by a process comprising crystallization of Acalabrutinib from a mixture comprising acetic acid as a solvent and methyl tert-butyl ether (“MTBE”) as an anti-solvent. The crystallization comprises providing a solution of Acalabrutinib in acetic acid and combining the solution with MTBE to obtain a gum-like material or a suspension.

Typically, the solution is provided at a temperature of from about 25° C. to about 30° C.

Combining the solution with the anti-solvent can be done by direct addition, i.e. the anti-solvent is added to the solution.

The process for preparing crystalline Form ACB3 of Acalabrutinib may in certain embodiments further comprise recovering said crystalline form. The recovery may be done, for example, by filtering the suspension, for example by vacuum filtration, and may optionally include washing and drying the crystalline form. Preferably, drying is done by air or by vacuum drying, typically at a temperature of from about 60° C. to about 70° C., for example for a period of from about 4 hours to about 24 hours.

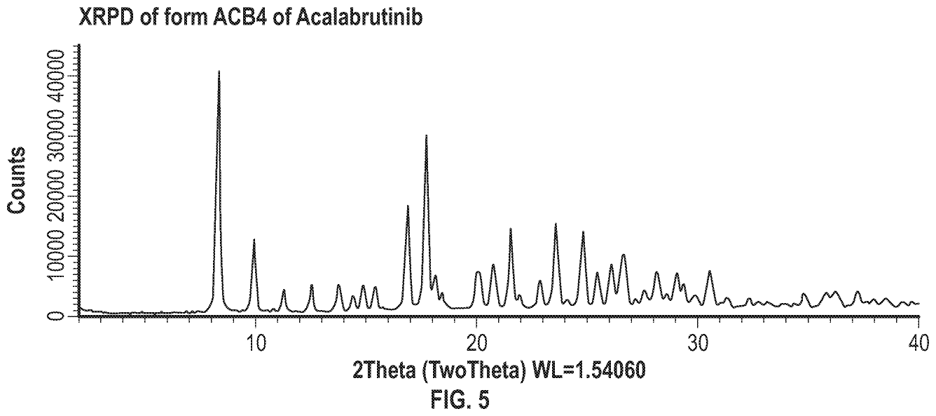

The present disclosure further includes a crystalline polymorph of Acalabrutinib designated Form ACB4. The crystalline Form ACB4 of Acalabrutinib may be characterized by data selected from one or more of the following: an X-ray powder diffraction pattern substantially as depicted in FIG. 5; an X-ray powder diffraction pattern having peaks at 8.4, 10.0, 17.0, 17.8, 21.7 and 23.7 degrees 2-theta±0.2 degrees 2-theta; and combinations of these data.

Crystalline Form ACB4 of Acalabrutinib may in some embodiments be further characterized by an X-ray powder diffraction pattern having peaks at 8.4, 10.0, 17.0, 17.8, 21.7 and 23.7 degrees 2-theta±0.2 degrees 2-theta, and also having any one, two, three, four or five additional peaks selected from 20.2, 20.9, 24.9 and 26.7 degrees 2-theta±0.2 degrees 2-theta.

Crystalline Form ACB4 of Acalabrutinib may be characterized by each of the above characteristics alone/or by all possible combinations, e.g., an XRPD pattern having peaks at 8.4, 10.0, 17.0, 17.8, 21.7 and 23.7 degrees 2-theta±0.2 degrees 2-theta; an XRPD pattern as depicted in FIG. 5, and combinations thereof.

In one embodiment of this aspect of the present disclosure, crystalline Form ACB4 of Acalabrutinib is isolated.

The crystalline Form ACB4 of Acalabrutinib may in certain embodiments be a methanol solvate. In some embodiments, the amount of methanol in crystalline form ACB4 may be from about 3% to about 7.5% (w/w), as measured by TGA.

Crystalline Form ACB4 of Acalabrutinib may be prepared by a process comprising precipitation of crystalline Form ACB4 from methanol. In some embodiments, the process comprises slurrying amorphous Acalabrutinib in methanol. Optionally, methyl tert-butyl ether (MTBE) can be added to the slurry.

The methanol may be aqueous methanol. In some embodiments, the methanol is from about 50% to about 95% aqueous methanol, meaning it contains from about 5% to about 50% (v/v) water.

The present disclosure also provides uses of the solid state forms of Acalabrutinib described herein for preparing other solid state forms of Acalabrutinib and/or Acalabrutinib co-crystals and salts, and their solid state forms.

The present disclosure thus also encompasses processes for preparing other solid state forms of Acalabrutinib and/or Acalabrutinib co-crystals and salts, and their solid state forms. Such processes include preparing a solid state form of the present disclosure, and converting it to other solid state forms of Acalabrutinib and/or Acalabrutinib co-crystals or salts, and their solid state forms.

In another aspect, the present disclosure encompasses the use of the above described solid state form of Acalabrutinib for the preparation of pharmaceutical compositions and/or pharmaceutical formulations. Such pharmaceutical compositions and/or pharmaceutical formulations may be suitable for the treatment of hematologic diseases, such as forms of blood cancers.

The present disclosure further provides pharmaceutical compositions comprising any one or a mixture of the solid state forms of Acalabrutinib according to the present disclosure. In some embodiments, the solid state form is Form ACB3.

In yet another embodiment, the present disclosure encompasses pharmaceutical formulations comprising any one or a mixture of the solid state form of Acalabrutinib (such as Form ACB3) and at least one pharmaceutically acceptable excipient.

Pharmaceutical formulations of the present invention contain any one or a combination of the crystalline forms of Acalabrutinib of the present disclosure. In some embodiments, the solid state form is Form ACB3.

The active ingredient and excipients can be formulated into compositions and dosage forms according to methods known in the art. In addition to the active ingredient, the pharmaceutical formulations of the present disclosure contain one or more pharmaceutically acceptable excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g.

Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present invention include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present disclosure, the active ingredient and any other solid excipients may be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present disclosure can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, the most preferred route of the present disclosure is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the invention, within either a hard or soft shell. The shell can be made from gelatin and optionally contain a plasticizer such as glycerin and sorbitol, and an opacifying agent or colorant.

A composition for tableting or capsule filling can for example be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, but they are not subjected to a final tableting step.

In some embodiments, a pharmaceutical formulation of Acalabrutinib is formulated for administration to a mammal, such as a human. Acalabrutinib can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others (Ansel et al., Pharmaceutical Dosage Forms and Drug Delivery Systems, 7th ed.).

The present disclosure further encompasses processes to prepare said formulations of Acalabrutinib. Such processes comprise combining any one or a mixture of the solid state forms of Acalabrutinib and at least one pharmaceutically acceptable excipient.

The solid state forms of Acalabrutinib as defined herein, as well as the pharmaceutical compositions or formulations thereof, can be used as medicaments, particularly for the treatment of hematologic diseases, such as forms of blood cancers. Examples of such blood cancers include chronic lymphocytic leukaemia (CLL), mantle cell lymphoma (MCL) and lymphoplasmacytic lymphoma (Waldenström's macroglobulinaemia, WM).

The present disclosure also provides methods of treating of hematologic diseases, such as forms of blood cancers; comprising administering a therapeutically effective amount of any one or a mixture of the solid state form of Acalabrutinib of the present disclosure, or at least one of the above pharmaceutical compositions or formulations, to a subject suffering from hematologic diseases (including forms of blood cancers), or otherwise in need of the treatment.

In another aspect, the present disclosure relates to (S)-4-(9-(1-(but-2-ynoyl)pyrrolidin-2-yl)-4-methyl-2-oxo-2H-imidazo[5′,1′:3,4]pyrazino[1,2-a]pyrimidin-11-yl)-N-(pyridin-2-yl)benzamide (“Compound 1”), which is an impurity of Acalabrutinib and to processes for its preparation.

Compound 1 has the following structure:

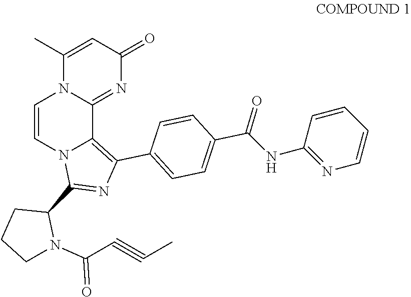 [SMILES: CC#CC(=O)N1CCC[C@H]1c1nc(-c2ccc(C(=O)Nc3ccccn3)cc2)c2c3nc(=O)cc(C)n3ccn12]

In some embodiments, Compound 1 can be isolated.

Compound 1 may be characterized by a mass spectrum M+H=532.2152. Compound 1 may also be characterized by the following ¹H-NMR or ¹³C-NMR peaks, as listed in Table 1:

TABLE 1

| SOLVENT | | DMSO-d6 | | |
|---|---|---|---|---|
| | ¹³C at 100 MHz | ¹H at 400 MHz | | |
| No | Chem. Shift, ppm | Chem. Shift, ppm | Multiplicity | J, Hz |
| 1 | 3.75. 3.84 | 2.02, 1.79 (6H) | s | ~ |
| 2 | 89.13, 88.64 | ~ | ~ | ~ |
| 3 | 74.60, 74.64 | ~ | ~ | ~ |
| 4 | 152.39, 152.24 | ~ | ~ | ~ |
| 5 | 48.81, 46.38 | 3.85 (2H) | t | 6.29 |
| 6 | 24.52, 23.21 | 2.37, 2.04 (2H) | m | ~ |
| 7 | 31.83, 32.87 | 2.37, 2.22 (2H) | m | ~ |
| 8 | 51.27, 53.82 | 5.55 (1H) | dd | 6.83, 4.16 |
| 9 | 144.95, 145.75 | ~ | ~ | ~ |
| 10 | 141.66, 141.98 | ~ | ~ | ~ |
| 11 | 117.00, 117.12 | ~ | ~ | ~ |
| 12 | 146.69, 146.63 | ~ | ~ | ~ |
| 13 | 113.32, 113.84 | 7.50, 7.52 (1H) | 2 × d | 6.46, 6.52 |
| 14 | 110.08, 109.39 | 8.06, 8.11 (1H) | 2 × d | 6.47, 6.55 |
| 15 | NH | | | ~ |
| 16 | 137.36, 137.25 | ~ | ~ | ~ |
| 17, 21 | 130.44 | 8.26, 8.30 (2H) | 2 × d | 8.62, 8.46 |
| 18, 20 | 127.55, 127.59 | 8.08, 8.09 (2H) | 2 × d | 8.45, 8.98 |
| 19 | 133.48, 133.53 | ~ | ~ | ~ |
| 22 | 166.17 | ~ | ~ | ~ |
| 23 | NH | 10.86 (1H) | s | ~ |
| 24 | 152.72 | ~ | ~ | ~ |
| 25 | 115.28 | 8.24 (1H) | d | 9.51 |
| 26 | 138.56 | 7.86 (1H) | td | 7.87, 1.75 |
| 27 | 120.27 | 7.18 (1H) | dd | 6.83, 5.25 |
| 28 | 148.42 | 8.41 (1H) | dd | 4.82, 1.06 |
| 29 | 167.45 | ~ | ~ | ~ |
| 30 | 112.76 | 6.20, 6.22 (1H) | 2 × s | ~ |
| 31 | 148.32 | ~ | ~ | ~ |
| 32 | 19.62 | 2.48 (3H) | s | ~ |

The above Compound 1 may be used as a reference standard in determining and/or quantifying the purity of Acalabrutinib.

Compound 1 may be prepared by a process comprising reacting Acalabrutinib with 2-butynoic acid, in the presence of a base, such as imidazole, trimethylamine, etc. To facilitate the reaction, a coupling agent may be used, such as pivaloyl chloride, hexafluorophosphate azabenzotriazole tetramethyl uronium (“HATU”) or 1-Ethyl-3-(3-dimethylaminopropyl)carbodiimide (EDCI).

The above reaction may be done in the presence of an organic solvent, for example a chlorinated solvent, such as dichloromethane, or non-chlorinated solvents, such as isopropyl acetate, tetrahydrofuran or toluene.

Compound 1 may be recovered from the reaction mixture, for example by extraction. In some embodiments, the process comprises adding water to the reaction mixture and adjusting the pH level to a level from which the organic layer and the aqueous layer separate. The aqueous layer may be subjected to additional separation step, by adding an organic solvent, such as dichloromethane, and adjusting the pH level. Compound 1 may be isolated from the organic layer by removing the solvents, for example by distillation or evaporation.

The pH level is typically adjusted to a strong acidic pH at the first separation step, for example a pH of about 1.8. At the second separation step, the pH level is typically adjusted to about neutral pH, for example in some embodiments to pH 6.9.

The isolated impurity Compound 1 can be purified, for example by HPLC.

In yet another aspect, the present disclosure relates to compositions comprising amorphous Acalabrutinib, and having the impurity Compound 1 at a level of less than about 0.2%; or less than about 0.15%; or less than about 0.1%.

In some embodiments, Compound 1 is present at a level of from about 0.02% to about 0.2%; or from about 0.02% to about 0.15; or from about 0.02% to about 0.1.

In other embodiments, Compound 1 is present at a level of from about 0.05% to about 0.2%; or from about 0.05% to about 0.15; or from about 0.05% to about 0.1.

In yet another aspect, the present disclosure relates to amorphous Acalabrutinib forming the respective impurity (Compound 1) at a level of less than about 0.2%; or less than about 0.15%; or less than about 0.1%, when stored at a temperature of about 25° C. and relative humidity (“RH”) of about 60% for a period of 1 month, or for a period of 3 months, or for a period of 6 months.

In some embodiments, the amorphous Acalabrutinib forms Compound 1 at a level of from about 0.02% to about 0.2%; or from about 0.02% to about 0.15; or from about 0.02% to about 0.1, when stored at a temperature of about 25° C. and RH of about 60% for a period of 1 month, or for a period of 3 months, or for a period of 6 months.

In certain embodiments, the amorphous Acalabrutinib forms Compound 1 at a level of from about 0.05% to about 0.2%; or from about 0.05% to about 0.15; or from about 0.05% to about 0.1, when stored at a temperature of about 25° C. and RH of about 60% for a period of 1 month, or for a period of 3 months, or for a period of 6 months.

Having described the disclosure with reference to certain preferred embodiments, other embodiments will become apparent to one skilled in the art from consideration of the specification. The disclosure is further illustrated by reference to the following examples describing in detail the preparation of the composition and methods of use of the disclosure. It will be apparent to those skilled in the art that many modifications, both to materials and methods, may be practiced without departing from the scope of the disclosure.

### Analytical Methods

### X-Ray Powder Diffraction Method:

Bruker D8 Advance; Copper Kα radiation (λ=1.5418 Å); Lynx eye detector; laboratory temperature 22-25° C.; PMMA specimen holder ring. Prior to analysis, the samples were gently ground by means of mortar and pestle in order to obtain a fine powder. The ground sample was adjusted into a cavity of the sample holder and the surface of the sample was smoothed by means of a cover glass. Silicon was used as a reference standard for determining peak positions.

### Measurement Parameters:

Scan range: 2-40 degrees 2-theta;
Scan mode: continuous;
Step size: 0.05 degrees;
Time per step: 0.5 s;
Sample spin: 30 rpm;
Sample holder: PMMA specimen holder ring.

### Solid State ¹³C-NMR Method:

Solid-state NMR spectra were measured at 11.7 T using a Bruker Avance III HD 500 US/WB NMR spectrometer (Karlsruhe, Germany, 2013) with 3.2 mm probehead. The 13C CP/MAS NMR spectra employing cross-polarization were acquired using the standard pulse scheme at spinning frequency of 15 kHz and a room temperature (298 K). The recycle delay was 8 s and the cross-polarization contact time was 2 ms. The 13C scale was referenced to α-glycine (176.03 ppm for 13C). Frictional heating of the spinning samples was offset by active cooling, and the temperature calibration was performed with Pb(NO3)2. The NMR spectrometer was completely calibrated and all experimental parameters were carefully optimized prior the investigation. Magic angle was set using KBr during standard optimization procedure and homogeneity of magnetic field was optimized using adamantane sample (resulting line-width at half-height Δν_{1/2} was less than 3.5 Hz at 250 ms of acquisition time).

### EXAMPLES

### Preparation of Starting Material

Acalabrutinib can be prepared by any process disclosed in the literature, for example in WO 2013/010868.

Acalabrutinib Form III can be prepared by any one of the processes described in WO 2017/002095.

Amorphous Acalabrutinib can be prepared by any known method for preparing amorphous materials, such as lyophilization, spray drying, fast evaporation, etc. Alternatively, it may be prepared by the process described herein below in Example 8, step i.

### Example 1: Preparation of Crystalline Acalabrutinib Form ACB1

Acalabrutinib (Form III, 0.1 grams) was added into a mixture of ethanol (0.25 ml) and methyl tert butylether (“MTBE”, 0.35 ml) at a temperature of 20° C.-30° C. and the obtained slurry was stirred for 3 days at the same temperature. The obtained solid was filtered under vacuum at a temperature of 20° C.-30° C. and was kept under suction for about 10-15 minutes. A sample was analyzed by PXRD, Form ACB1 was obtained (0.07 grams).

### Example 2: Preparation of Crystalline Acalabrutinib Form ACB1

Acalabrutinib (Form III, 0.07 grams) was dissolved in ethanol (0.5 ml) at a temperature of 30-50° C., and was stirred for 5-10 minutes to obtain a clear solution. n-Heptane (1.5 ml, pre-maintained at a temperature of 20° C.-30° C.) was added into the clear solution, under magnetic stirring and a gummy material was obtained and was stirred for 24 hours at a temperature of 20° C.-30° C. The obtained solid was filtered under vacuum at a temperature of 20° C.-30° C. and kept under suction for about 10-15 minutes. A sample was analyzed by PXRD, Form ACB1 was obtained (0.05 grams).

### Example 3: Preparation of Crystalline Acalabrutinib Form ACB1

Acalabrutinib (Form III, 0.07 grams) was dissolved in ethanol (0.5 ml) at a temperature of 30° C.-50° C. and was stirred for 5-10 minutes to obtain a clear solution. The obtained clear solution was added into n-heptane (1.5 ml, pre-maintained at a temperature of 20° C.-30° C.) under magnetic stirring and a gummy material was obtained and was stirred for 24 hours at a temperature of 20° C.-30° C. The obtained solid was filtered under vacuum at 20° C.-30° C. and was kept under suction for about 10-15 minutes. A sample was analyzed by PXRD, Form ACB1 was obtained (0.05 grams).

### Example 4: Preparation of Crystalline Acalabrutinib Form ACB1

Acalabrutinib (Form III, 0.07 grams) was dissolved in ethanol (0.5 ml) at a temperature of 30° C.-50° C. and was stirred for 5-10 minutes to obtain a clear solution. n-heptane (1.5 ml, pre-maintained at a temperature of 30° C.-50° C.) was added into the clear solution under magnetic stirring and a gummy material was obtained and was stirred for 24 hours at a temperature of 30° C.-50° C. The obtained solid was filtered under vacuum at a temperature of 20° C.-30° C. and was kept under suction for about 10-15 minutes. A sample was analyzed by PXRD, Form ACB1 was obtained (0.05 grams). A PXRD pattern is shown in FIG. 1.

### Example 5: Preparation of Crystalline Acalabrutinib Form ACB1

Acalabrutinib (Form III, 0.07 grams) was dissolved in ethanol (0.5 ml) at a temperature of 30° C.-50° C. and was stirred for 5-10 minutes to obtain a clear solution. The obtained clear solution was added into n-heptane (1.5 ml, pre-maintained at a temperature of 30°−50° C.) under magnetic stirring and a gummy material was obtained and was stirred for 24 hours at a temperature of 30° C.-50° C. The obtained solid was filtered under vacuum at 20° C.-30° C. and was kept under suction for about 10-15 minutes. A sample was analyzed by PXRD, Form ACB1 was obtained (0.05 grams).

### Example 6: Preparation of Crystalline Acalabrutinib Form ACB2

Acalabrutinib (Form III, 0.1 grams) was added into acetonitrile (1.5 ml, moisture content less than 1%) at a temperature of 40° C.-50° C. and was stirred for 10-20 minutes at the same temperature to obtain a clear solution. The obtained clear solution was kept under stirring at a temperature of 0° C.-5° C. for 5 days. The obtained solid was filtered under vacuum at a temperature of 0° C.-5° C. and was kept under suction for about 10-15 minutes at a temperature of 20° C.-30° C. The solid was further dried in ATD (air tray drier) at a temperature of 140° C. for 1 hour. A sample was analyzed by PXRD, Form ACB2 was obtained (0.07 g).

### Example 7: Preparation of Crystalline Acalabrutinib Form ACB2

Acalabrutinib (Form III, 0.5 grams) was added into acetonitrile (1.5 ml, moisture content less than 1%) at a temperature of 40° C.-50° C. and was stirred for 10-20 minutes at the same temperature, and a gummy-sticky solid was formed. Additional amount of acetonitrile (3.5 ml) was added and the mixture was kept under stirring at a temperature of 45° C. for 1-3 hours. The obtained solid was filtered under vacuum at a temperature of 20° C.-25° C. and was kept under suction for about 10-15 minutes at a temperature of 20° C.-30° C. The solid was further dried in ATD (air tray drier) at a temperature of 140° C. for 1 hour. A sample was analyzed by PXRD, Form ACB2 was obtained (0.35 grams). A PXRD pattern is shown in FIG. 2.

### Example 8: Preparation of Crystalline Acalabrutinib Form ACB2

Acalabrutinib (Amorphous, 0.1 gm) was added into a 10 ml vial containing a mixture of Acetonitrile and water (4 ml, 95:5 ratio) at a temperature of about 25-30° C. The mixture was stirred for 10 minutes at same temperature and the obtained slurry was heated to a temperature of about 50° C. and maintained for about 5-10 minutes at this temperature to form a clear solution. The clear solution was then cooled down to a temperature of about 25-30° C. over a period of about 5-10 minutes, and it was left to crystallize at the same temperature. After 10 days, the obtained solid was filtered under vacuum and kept under suction for about 10-15 minutes at a temperature of about 20-30° C. to obtain crystalline Acalabrutinib. A sample was analyzed by PXRD, Form ACB2 was obtained.

### Example 9: Preparation of Crystalline Acalabrutinib Form ACB3

### Step i: Preparation of Crude Acalabrutinib

2-Butynoic acid (2.1 grams), imidazole (2.13 grams) and dichloromethane (100 ml) were mixed under stirring for 30 minutes at a temperature of about 20-30° C., then the reaction mixture was cooled to temperature of about 0 to about 10° C. and further stirred for 10 minutes. Pivaloyl chloride (2.26 grams) was added over a period of 5 minutes, and the mixture was stirred for 1 hour at the same temperature. Then, (S)-4-(8-amino-3-(pyrrolidin-2-yl)imidazo[1,5-a]pyrazin-1-yl)-N-(pyridin-2-yl)benzamide, (5.0 grams) was added and the mixture was stirred for 30 minutes, the reaction completion was monitored by HPLC. Water (50 ml) was added at a temperature of about 5-10° C., and the mixture was stirred for 30-45 minutes at a temperature of about 10-25° C. The pH was adjusted to pH 5.9 using aqueous sodium carbonate solution (1 gram of sodium carbonate in 10 ml water), and the mixture was stirred for 10-15 minutes. Then the layers were separated and the organic layer was collected. water (50 ml) was added and the pH was adjusted to pH 1-1.1 using ˜35% conc. HCl solution (approx. 6 ml) at a temperature of about 15-25° C. The mixture was stirred for 15 minutes then the layers were separated and the aqueous layer was collected. The aqueous layer was washed twice with dichloromethane (25 ml) stirred at a temperature of about 15-25° C. for 30 minutes, then layers were separated and the aqueous layer was collected. Dichloromethane (50 ml) was added to the aqueous layer and then the pH was adjusted to pH 6.5 using aqueous sodium carbonate solution (2 grams of sodium carbonate and 20 ml water) at a temperature of about 15-25° C. The mixture was stirred for 30 minutes then layers were separated and the organic layer was collected. Water (50 ml) was added and the mixture was stirred for 20-30 minutes at a temperature of about 15-25° C. then layers were separated and the final organic layer was collected. The solvent was distilled off under vacuum at a temperature of about 38° C., crude Acalabrutinib residue was obtained (6.2 grams, amorphous)

### Step ii: Preparation of Crystalline Acalabrutinib Form ACB3

Premixed mixture of acetone (7.5 ml) and n-heptane (22.5 ml) was added to Acalabrutinib crude residue (2 grams, amorphous) at a temperature of about 25° C. The mass was heated to a temperature of about 50-55° C. and was stirred for 60-90 minutes. Water (0.16 ml) was added and the mixture was stirred for 30 minutes at the same temperature. Then, gradually the mixture was cooled down to a temperature of about 25-30° C. over a period of about 40 minutes and maintained for 15 minutes. The obtained solid was filtered under vacuum at a temperature of about 20-30° C. and washed with n-heptane (4 ml), then kept under suction for about 10-15 minutes at a temperature of about 20-30° C. The obtained solid was dried in a vacuum tray drier, under vacuum at a temperature of about 45-50° C. for a period of 3 hours. A sample was analyzed by PXRD, Form ACB3 was obtained (yield: 1.35 grams). A PXRD pattern is shown in FIG. 4.

### Example 10: Preparation of Crystalline Acalabrutinib Form ACB3

Acalabrutinib crude residue (2 grams, amorphous) was added to ethanol (8 ml) at a temperature of about 25° C. The mass was heated to a temperature of about 54° C. and n-heptane (4 ml) was added slowly over a period of 10 minutes. The mixture was stirred for 2 hours at the same temperature, then, it was gradually cooled down to a temperature of about 20-25° C. over a period of 1 hour and maintained for 60 minutes at the same temperature. The obtained solid was filtered under vacuum at a temperature of about 20-30° C. and was kept under suction for about 10-15 minutes at a temperature of about 20-30° C. The solid was further dried in a vacuum tray drier under vacuum at a temperature of about 25° C. for 3 hours. A sample was analyzed by PXRD, Form ACB3 was obtained (yield: 1.4 grams).

### Example 11: Preparation of Crystalline Acalabrutinib Form ACB3

### Step i: Preparation of Crude Acalabrutinib

2-Butynoic acid (25.25 grams), imidazole (25.6 grams) and dichloromethane (1200 ml) were mixed under stirring for 5 minutes at a temperature of about 20-30° C. Then, the reaction mixture was cooled to a temperature of about 0-10° C. and was stirred for 15 minutes. Then, pivaloyl chloride (27.17 grams) was added slowly over a period of 15 minutes, and the reaction mixture was stirred for 1 hour at the same temperature. (S)-4-(8-amino-3-(pyrrolidin-2-yl)imidazo[1,5-a]pyrazin-1-yl)-N-(pyridin-2-yl)benzamide (60 grams) was added and the reaction mixture was stirred for 30 minutes and the reaction completion was monitored by HPLC. 600 ml of water was added at a temperature of about 0-25° C. and the mixture was stirred for 30-45 minutes at a temperature of about 15-25° C. The layers were separated, the organic layer was collected at a temperature of about 15-25° C., 600 ml of water was added and the pH was adjusted to pH 0.8-1.1 with ˜35% conc. HCl solution (approx. 35 ml) at a temperature of about 15-25° C. The mixture was stirred for 45 minutes and the layers were separated and the aqueous layer was collected. Dichloromethane (600 ml) was added to the aqueous layer and it was stirred for 30 minutes at a temperature of about 15-25° C. The layers were separated and the aqueous layer was collected. Dichloromethane (600 ml) was added to the aqueous layer and the pH was adjusted to pH 6.5-7.0 using aqueous sodium carbonate solution (18.5 grams of sodium carbonate and 185 ml water) at a temperature of about 15-25° C., and the mixture was stirred for 30 minutes. The layers were separated, the organic layer at was collected, then water (300 ml) was added; the mixture was stirred for 20-30 minutes at a temperature of about 15-25° C. The layers were separated, and the final organic layer was collected. The solvent was distilled off under vacuum at a temperature of about 40° C., and crude Acalabrutinib residue was obtained (75 grams, amorphous).

### Step ii: Preparation of Crystalline Acalabrutinib Form ACB3

Acalabrutinib crude residue (75 grams, amorphous) was dissolved in 300 ml ethanol (300 ml) (at a temperature of about 50-55° C. and a clear solution was formed. n-heptane (150 ml) was added to the clear solution over a period of about 20 minutes, and the solution was stirred for 10 minutes at the same temperature. Then, the clear solution was gradually cooled down to a temperature of about 20° C. over a period of 5 hours, and the slurry was maintained for 30 minutes at the same temperature and a solid was formed. The obtained solid was filtered under vacuum at a temperature of about 20-30° C., then washed with a mixture of ethanol:n-heptane (2:1; 120 ml). The washed solid was maintained under suction for about 10-15 minutes at 20-30° C. and further dried the compound in a VTD (vacuum tray drier) under vacuum at a temperature of about 40-50° C. for 25 hours. Crystalline Acalabrutinib was obtained (48 grams). A sample was analyzed by PXRD, Form ACB3 was obtained.

### Example 12: Preparation of Crystalline Acalabrutinib Form ACB3

Acalabrutinib (amorphous, 1 g) and acetic acid (5 ml) were mixed in a 500 ml round bottom flask at a temperature of about 25-30° C., and a clear solution formed. MTBE (110 ml) was added to the clear solution and a gel/gummy like material was formed. It was further maintained upon stirring for a period of about 24 hours at the same temperature. The obtained solid was filtered under vacuum at a temperature of about 15-30° C. and the isolated material was kept under suction for about 20-30 minutes at a temperature of about 20-30° C. Then, it was washed with MTBE (10 ml) and further dried in an air tray drier (ATD) at a temperature of about 70° C. for a period of about 4 hours. Crystalline Acalabrutinib was obtained (0.7 grams). A sample was analyzed by PXRD, Form ACB3 was obtained.

### Example 13: Preparation of Crystalline Acalabrutinib Form ACB3

Acalabrutinib (amorphous, 2 grams) and acetic acid (5 ml) were mixed in a 500 ml round bottom flask at a temperature of about 25-30° C., and a clear solution formed. MTBE (220 ml) was added to the clear solution and the mixture maintained upon stirring for a period of about 72 hours at the same temperature. The obtained solid was filtered under vacuum at a temperature of about 15-30° C. and the isolated material kept under suction for about 20-30 minutes at a temperature of about 20-30° C. Then, the material was washed with MTBE (10 ml) and further dried in a vacuum tray drier (VTD) at a temperature of about 60° C. for a period of about 40 hours. Crystalline Acalabrutinib was obtained (1.6 grams). A sample was analyzed by PXRD, Form ACB3 was obtained.

### Example 14: Preparation of Crystalline Acalabrutinib Form ACB3

Acalabrutinib (Form I, 0.200 grams) was mixed with a mixture of MTBE and acetic acid (90% MTBE, 10% acetic acid, total volume 4 ml) in a 5 ml vial and the obtained slurry were stirred for 6 hours at a temperature of about 15-30° C. The obtained solid was filtered under vacuum at a temperature of about 15-30° C. and kept under suction for about 10-15 minutes at a temperature of about 20-30° C. Then, the filtered material was washed with MTBE (10 ml) and further dried in an ATD at a temperature of about 70° C. for 6 hours to obtain crystalline Acalabrutinib (0.12 grams). A sample was analyzed by PXRD, Form ACB3 was obtained.

### Example 15: Preparation of Crystalline Acalabrutinib Form ACB3

Acalabrutinib (Form I, 1.5 grams) was mixed with a mixture of MTBE and acetic acid (60% MTBE, 40% acetic acid, total volume 5 ml) in a 10 ml vial, and an additional amount of MTBE (2 ml) was added. The obtained slurry were stirred for 48 hours at a temperature of about 15-30° C. The obtained solid was filtered under vacuum at 15-30° C. and kept under suction for about 10-15 minutes at a temperature of about 20-30° C. The filtered solid was washed with MTBE (10 ml) and further dried in an ATD at a temperature of about 70° C. for 6 hours to obtain crystalline Acalabrutinib (1.0 gram). A sample was analyzed by PXRD, Form ACB3 was obtained.

### Example 16: Preparation of Crystalline Acalabrutinib Form ACB4

Acalabrutinib (amorphous form, 0.740 grams) was added into a 2 ml vial with aqueous methanol (95%, 1.4 ml) and the obtained slurry was stirred for 24 hours at a temperature of 0-5° C. The obtained solid was filtered under vacuum at a temperature of 15-30° C. and kept under suction for a period of about 5-10 minutes at a temperature of 20-30° C. to obtain crystalline Acalabrutinib (0.9 grams). A sample was analyzed by PXRD, form ACB4 was obtained.

### Example 17: Preparation of Crystalline Acalabrutinib Form ACB4

Acalabrutinib (amorphous form, 0.740 grams) was added into a 2 ml vial with aqueous methanol (95%, 0.7 ml) and the obtained slurry was stirred for 24 hours at a temperature of 0-5° C. The obtained solid was filtered under vacuum at a temperature of 15-30° C. and kept under suction for a period of about 5-10 minutes at a temperature of 20-30° C. to obtain crystalline Acalabrutinib (0.9 grams). A sample was analyzed by PXRD, form ACB4 was obtained. A PXRD pattern is shown in FIG. 5.

### Example 18: Preparation of Crystalline Acalabrutinib Form ACB4

Acalabrutinib (amorphous form, 0.5 grams) was added into a 2 ml vial with aqueous methanol (80%, 0.5-0.9 ml) and the obtained slurry was stirred for 24 hours at a temperature of 0-5° C. The obtained solid was filtered under vacuum at a temperature of 15-30° C. and kept under suction for a period of about 5-10 minutes at a temperature of 20-30° C. to obtain crystalline Acalabrutinib (0.65 grams). A sample was analyzed by PXRD, form ACB4 was obtained.

### Example 19: Preparation of Crystalline Acalabrutinib Form ACB4

Acalabrutinib (amorphous form, 0.5 grams) was added into a 2 ml vial with aqueous methanol (75%, 0.5-0.9 ml) and the obtained slurry was stirred for 24 hours at a temperature of 0-5° C. The obtained solid was filtered under vacuum at a temperature of 15-30° C. and kept under suction for a period of about 5-10 minutes at a temperature of 20-30° C. to obtain crystalline Acalabrutinib (0.65 grams). A sample was analyzed by PXRD, form ACB4 was obtained.

### Example 20: Preparation of Crystalline Acalabrutinib Form ACB4

Acalabrutinib (amorphous form, 0.5 grams) was added into a 2 ml vial with aqueous methanol (65%, 0.5-0.9 ml) and the obtained slurry was stirred for 24 hours at a temperature of 0-5° C. The obtained solid was filtered under vacuum at a temperature of 15-30° C. and kept under suction for a period of about 5-10 minutes at a temperature of 20-30° C. to obtain crystalline Acalabrutinib (0.65 grams). A sample was analyzed by PXRD, form ACB4 was obtained.

### Example 21: Preparation of Crystalline Acalabrutinib Form ACB4

Acalabrutinib (amorphous form, 0.5 grams) was added into a 2 ml vial with aqueous methanol (50%, 0.5-0.9 ml) and the obtained slurry was stirred for 24 hours at a temperature of 0-5° C. The obtained solid was filtered under vacuum at a temperature of 15-30° C. and kept under suction for a period of about 5-10 minutes at a temperature of 20-30° C. to obtain crystalline Acalabrutinib (0.65 grams). A sample was analyzed by PXRD, form ACB4 was obtained.

### Example 22: Preparation of Crystalline Acalabrutinib Form ACB4

Acalabrutinib (amorphous form, 0.5 grams) was added into a 10 ml vial with aqueous methanol (95%, 0.5 ml) and the obtained slurry was stirred for 2 hours at a temperature of 0-5° C. Then, MTBE (3.5 ml) was added to the slurry and it was further stirred for 24 hours at a temperature of 0-5° C. The obtained solid was filtered under vacuum at a temperature of 15-30° C. and kept under suction for a period of about 5-10 minutes at a temperature of 20-30° C. to obtain crystalline Acalabrutinib (0.55 grams). A sample was analyzed by PXRD, form ACB4 was obtained.

### Example 23: Preparation of Amorphous Acalabrutinib

Acalabrutinib pure (40 grams), prepared and isolated according to Example 10 steps i) and ii), was dissolved in methanol (240 ml) at a temperature of about 20-25° C. and a clear solution formed. The solvent was distilled of at a temperature of about 40-45° C. (Tj) afforded a solid (40.6 grams), which was dried under vacuum at a temperature of about 40-45° C. (Tj) to afford amorphous Acalabrutinib (37.5 grams).

### Example 24: Preparation of (S)-4-(9-(1-(but-2-ynoyl)pyrrolidin-2-yl)-4-methyl-2-oxo-2H-imidazo[5′,1′:3,4]pyrazino[1,2-a]pyrimidin-11-yl)-N-(pyridin-2-yl)benzamide—Compound 1

2-Butynoic acid (1.5 grams), imidazole (1.5 grams) and dichloromethane (30 ml) were mixed under stirring for 30 minutes at a temperature of about 20-25° C. Pivaloyl chloride (2.2 grams) was added, followed by Acalabrutinib (3.0 grams). The reaction mixture was stirred for 15 hours at a temperature of about 20-25° C. and the reaction progress was monitored by HPLC. Water (20 ml) was added at a temperature of about 20-25° C., and the mixture was stirred for 30 minutes. The pH was adjusted to 1.8 using concentrated HCl (1.8 ml) and the mixture was stirred for 10-15 minutes. The layers were separated and the aqueous layer was collected. Dichloromethane (15 ml) was added to the aqueous layer and the pH was adjusted to 6.9 using ˜20% aqueous sodium carbonate (approx. 8 ml) at a temperature of about 20-25° C. The mixture was stirred for 15 minutes, then the layers were separated and the organic layer was collected. The solvent was distilled under vacuum at a temperature of about 35° C. (Tj) to obtain crude Compound 1 (3.1 grams) having an HPLC purity of 59.74%, which was isolated by prep. HPLC to afford 100 mg of Compound 1 with an HPLC purity of 99.59%.

1. Crystalline Form ACB3 of Acalabrutinib characterized by data selected from one or more of the following:
a) an XRPD pattern having peaks at 6.3, 16.3, 17.5, 18.5, 19.6 and 24.0 degrees 2-theta±0.2 degrees 2-theta;
b) an XRPD pattern as depicted in FIG. 4;
c) a ¹³C solid state NMR having peaks in the range of 100-200 ppm at 107.0, 113.8, 137.8, 141.9, 146.5 and 165.4 ppm±0.2 ppm;
d) a solid state ¹³C NMR spectrum having absolute chemical shift differences from a reference peak at 127.3±2 ppm of 20.3, 13.5, 10.5, 14.6, 19.2 and 38.1 ppm±0.1 ppm respectively;
e) a ¹³C solid state NMR spectrum substantially as depicted in FIG. 6a, 6b or 6c; and/or
f) combinations of these data.

2. Crystalline Form ACB3 of Acalabrutinib according to claim 1, characterized by an XRPD pattern having peaks at 6.3, 16.3, 17.5, 18.5, 19.6 and 24.0 degrees 2-theta±0.2 degrees 2-theta, and also having one, two, three, four or five additional peaks selected from 10.3, 13.1, 15.1, 20.5 and 27.7 degrees two theta±0.2 degrees two theta.

3. Crystalline Form ACB3 of Acalabrutinib according to claim 1, wherein said crystalline form is an anhydrous form.

4. Crystalline Form ACB3 of Acalabrutinib according to claim 1, which contains no more than about 20 wt % of any other crystalline forms of Acalabrutinib.

5. (canceled)

6. A pharmaceutical composition comprising crystalline Form ACB3 of Acalabrutinib according to claim 1.

7. A pharmaceutical formulation comprising crystalline Form ACB3 of Acalabrutinib according to claim 1, and at least one pharmaceutically acceptable excipient.

8. A process for preparing a pharmaceutical formulation comprising combining a crystalline Form ACB3 of Acalabrutinib according to claim 1 with at least one pharmaceutically acceptable excipient.

9. A medicament comprising the crystalline Form ACB3 of Acalabrutinib according to claim 1.

10. (canceled)

11. A method of treating hematologic diseases, optionally wherein the hematologic disease is a form of blood cancer, comprising administering a therapeutically effective amount of crystalline Form ACB3 of Acalabrutinib according to claim 1 to a subject in need of the treatment.

12. (canceled)

13. A process for preparing an Acalabrutinib salt or a solid state form thereof, comprising preparing crystalline Form ACB3 of Acalabrutinib according to claim 1, and converting it to an Acalabrutinib salt, co-crystal or a solid state form thereof.

14. (S)-4-(9-(1-(but-2-ynoyl)pyrrolidin-2-yl)-4-methyl-2-oxo-2H-imidazo[5′,1′:3,4]pyrazino[1,2-a]pyrimidin-11-yl)-N-(pyridin-2-yl)benzamide (“Compound 1”) having the formula:

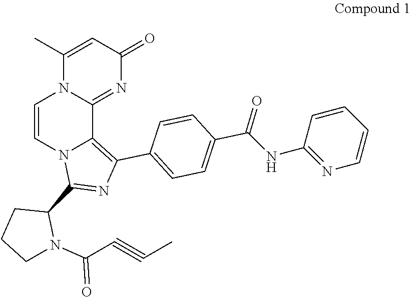 [SMILES: CC#CC(=O)N1CCC[C@H]1c1nc(-c2ccc(C(=O)Nc3ccccn3)cc2)c2c3nc(=O)cc(C)n3ccn12]

15. The compound according to claim 14 in isolated form.

16. A composition comprising amorphous Acalabrutinib, and the compound according to claim 15 as an impurity.

17. The composition of claim 16, wherein Compound 1 is present at a level of less than about 0.2 wt %.

18. The composition of claim 17, wherein Compound 1 is present at a level of from about 0.02 wt % to about 0.2 wt %.

19. The composition of claim 17, wherein Compound 1 is present at a level of from about 0.05 wt % to about 0.2 wt %.

20. Amorphous Acalabrutinib including the compound according to claim 14 at a level of less than about 0.2 wt % when stored at a temperature of about 25° C. and relative humidity (“RH”) of about 60% for a period of 1 month.

21. Amorphous Acalabrutinib according to claim 20, wherein the compound of claim 14 is formed at a level of from about 0.02 wt % to about 0.2 wt % when stored at a temperature of about 25° C. and RH of about 60% for a period of 1 month.

22. Amorphous Acalabrutinib according to claim 20, wherein the compound of claim 14 is formed at a level of from about 0.05 wt % to about 0.2 wt % when stored at a temperature of about 25° C. and RH of about 60% for a period of 1 month.
